(19) United States
(12) Patent Application Publication
(10) Pub. No.: US 2021/0251877 A1
(43) Pub. Date: Aug. 19, 2021

(54) TWO-STEP REACTIVE LIP SYSTEM

(71) Applicant: L'ORÉAL, Paris (FR)

(72) Inventors: Jody EBANKS, Clark, NJ (US); Chunhua LI, Hillsborough, NJ (US); Tsang-Min HUANG, Scotch Plains, NJ (US); Julien PORTAL, Les Pavillons-sous-Bois (FR)

(73) Assignee: L'ORÉAL, Paris (FR)

(21) Appl. No.: 16/794,934

(22) Filed: Feb. 19, 2020

(51) Int. Cl. A61K 8/898; A61K 8/37; A61K 8/31; A61Q 1/06

(52) U.S. Cl. CPC A61K 8/898; A61Q 1/06; A61K 8/31; A61K 8/37

(57) ABSTRACT

Disclosed is a two-step cosmetic product, such as used on the lips, where the basecoat includes a surface-stabilized dispersion of copolymer particles, and the topcoat comprises a silicone amine and a viscous silicone, the viscous silicone having a viscosity of at least about 100,000 centistoke when measured at a concentration by weight of 100% and using a rotational viscometer at 25° C.

### FIELD OF THE INVENTION

The present invention relates to cosmetic compositions, and specifically to two-step cosmetics, where the basecoat includes a surface-stabilized dispersion of copolymer particles, and the topcoat includes a silicone amine and a viscous silicone.

### BACKGROUND

Cosmetic products such as lip glosses and other lip cosmetics are expected to have good wear and transfer resistance properties, while still being non-sticky and having a glossy finish. However, this combination of characteristics is challenging to satisfy, especially for products that are also intended to make the lips look fuller and/or plumper upon application. Therefore, a cosmetic product that can provide this combination of characteristics is useful and desirable.

### BRIEF SUMMARY

A first aspect of the present disclosure is a two-step reactive lip system, comprising a basecoat and a topcoat. The base coat is applied to the lips first, and the topcoat is then applied onto the base coat. The basecoat includes a dispersion of copolymer particles surface-stabilized with one or more stabilizers in a non-aqueous medium containing at least one hydrocarbon-based oil, where the copolymer is a C₁-C₄ alkyl (meth)acrylate and maleic anhydride. The topcoat includes a silicone amine and a viscous silicone, the viscous silicone has a viscosity of at least about 100,000 centistoke when measured at a concentration by weight of 100% and using a rotational viscometer at 25° C.

Optionally, the copolymer particles and stabilizers contain between about 10% and about 30% by weight of the one or more stabilizers, between about 50% and about 89.99% by weight of the C₁-C₄ alkyl (meth)acrylate, and between about 0.01% and about 25% of the maleic anhydride. Optionally, the copolymer particles are present in the dispersion in an amount of between about 20% and about 60% by weight of the total dispersion.

The copolymer may optionally contain between 80% and 99.99% by weight of the C₁-C₄ alkyl (meth)acrylate and between 0.01% and 20% by weight of the maleic anhydride.

The one or more stabilizers may include a (C₃-C₁₂)cycloalkyl (meth)acrylate polymer, the (C₃-C₁₂)cycloalkyl (meth)acrylate polymer being a (C₃-C₁₂)cycloalkyl (meth)acrylate homopolymer or a statistical copolymer of (C₃-C₁₂)cycloalkyl (meth)acrylate and of C₁-C₄ alkyl (meth)acrylate present in a (C₃-C₁₂)cycloalkyl (meth)acrylate/C₁-C₄ alkyl (meth)acrylate weight ratio of greater than 4. In some systems, the one or more stabilizers comprises isobornyl acrylate. In some systems, only a single stabilizer is utilized.

Optionally, the hydrocarbon oil that is part of the non-aqueous medium the surface-stabilized copolymer particles are dispersed within contains between 10 and 18 carbons, such as n-decane, isodecane, isododecane, n-tetradecane, or isohexadecane.

The base coat may also contain other materials, such as at least one pigment, which may be present in the base coat in an amount of between 0.5% and 40% by weight.

With the top coat, the silicone amine may optionally be present in a total amount of between 0.5% and 40% by weight in the top coat composition.

Optionally, the viscous silicone is a dimethiconol. Optionally, the viscous silicone is present in a total amount of between about 0.1% and about 50% by weight in the top coat composition.

A second aspect of the present disclosure is a method for providing long wear in a lip cosmetic. The method involves applying a basecoat, then applying a top coat to the basecoat. The base coat is applied to the lips first, and the topcoat is then applied onto the base coat. The basecoat includes a dispersion of copolymer particles surface-stabilized with one or more stabilizers in a non-aqueous medium containing at least one hydrocarbon-based oil, where the copolymer is a C₁-C₄ alkyl (meth)acrylate and maleic anhydride. The topcoat includes a silicone amine and a viscous silicone, the viscous silicone has a viscosity of at least about 100,000 centistoke when measured at a concentration by weight of 100% and using a rotational viscometer at 25° C.

Optionally, the styrenic block copolymer is a styrene and ethylene/butylene triblock copolymer. The styrenic block copolymer may be present in the base coat in an amount between about 1% and about 20% by weight in the base coat composition.

Optionally, the copolymer particles and stabilizers contain between about 10% and about 30% by weight of the one or more stabilizers, between about 50% and about 89.99% by weight of the C₁-C₄ alkyl (meth)acrylate, and between about 0.01% and about 25% of the maleic anhydride. Optionally, the copolymer particles are present in the dispersion in an amount of between about 20% and about 60% by weight of the total dispersion.

The copolymer may optionally contain between 80% and 99.99% by weight of the C₁-C₄ alkyl (meth)acrylate and between 0.01% and 20% by weight of the maleic anhydride.

The one or more stabilizers may include a (C₃-C₁₂)cycloalkyl (meth)acrylate polymer, the (C₃-C₁₂)cycloalkyl (meth)acrylate polymer being a (C₃-C₁₂)cycloalkyl (meth)acrylate homopolymer or a statistical copolymer of (C₃-C₁₂)cycloalkyl (meth)acrylate and of C₁-C₄ alkyl (meth)acrylate present in a (C₃-C₁₂)cycloalkyl (meth)acrylate/C₁-C₄ alkyl (meth)acrylate weight ratio of greater than 4. In some systems, the one or more stabilizers comprises isobornyl acrylate. In some systems, only a single stabilizer is utilized.

Optionally, the hydrocarbon oil that is part of the non-aqueous medium the surface-stabilized copolymer particles are dispersed within contains between 10 and 18 carbons, such as n-decane, isodecane, isododecane, n-tetradecane, or isohexadecane.

The base coat may also contain other materials, such as at least one pigment, which may be present in the base coat in an amount of between 0.5% and 40% by weight.

With the top coat, the silicone amine may optionally be present in a total amount of between 0.5% and 40% by weight in the top coat composition.

Optionally, the viscous silicone is a dimethiconol. Optionally, the viscous silicone is present in a total amount of between about 0.1% and about 50% by weight in the top coat composition.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “alkyl radical” is a saturated hydrocarbon group in C₁-C₈, linear or branched, in particular in C₁-C₆, preferably in C₁-C₄ such as methyl, ethyl, isopropyl, and t-butyl.

As used herein, an “alkylene radical” is a saturated hydrocarbon group divalent in C₁-C₈, linear or branched, in particular in C₁-C₆, preferably in C₁-C₄ such as methylene, ethylene, or propylene;

As used herein, the term “anhydrous” dispersion or composition refers to a dispersion or composition containing less than 2% by weight of water, or even less than 0.5% of water, and notably free of water. Where appropriate, such small amounts of water may notably be introduced by ingredients of the composition that may contain residual amounts thereof.

As used herein, an “aryl radical” is a cyclic hydrocarbon-based radical, aromatic unsaturated, comprising from 6 to 12 carbon atoms, mono or bicyclic, fused or not, preferably the aryl group comprises 1 ring and with 6 carbon atoms such as phenyl.

As used herein, the term “aryloxy radical” means an aryl-oxy radical, i.e., aryl-O— with aryl as defined above, preferably phenoxy.

As used herein, the term “aryl (C1-C4) alkoxy radical” means an aryl-(C1-C4) alkyl-O-radical, preferably benzoxy.

As used herein, the term “at least one” means one or more and thus includes individual components as well as mixtures/combinations.

As used herein, the term “between [two numbers]” is intended to include those two numbers. For example, “x is between 1 and 2” is intended to cover 1≤x≤2. The term “x is between about 1 and about 2” would be intended to cover 0.95≤x≤2.05.

As used herein, the term “cyclic radical” means a cyclic, saturated or unsaturated, aromatic or non-aromatic hydrocarbon group, comprising from 1 to 3 rings, preferably 1 ring, and comprising from 3 to 10 carbon atoms such as cyclohexyl or phenyl.

As used herein, the term “cycloalkyl radical” means a saturated cyclic hydrocarbon group comprising from 1 to 3 rings, preferably 2 rings, and comprising from 3 to 13 carbon atoms, preferably between 5 and 10 carbon atoms, such as cyclopentyl, cyclohexyl, cycloheptyl, norbornyl, or isobornyl, the cycloalkyl radical potentially being substituted by one or more (C₁-C₄) alkyl groups such as methyl, preferably the cycloalkyl radical is an isobornyl group.

As used herein, the term “ethylenic copolymer” means a polymer resulting from the polymerization of two monomers: of the monomer a) (C₁-C₄)alkyl (C₁-C₄)(alkyl)acrylate and of the monomer b) of ethylenically unsaturated anhydride compound.

As used herein, the term “ethylenically unsaturated anhydride compound” means a carboxylic acid anhydride compound comprising at least one ethylenic unsaturation —(Rₐ)C═C(R_{b})—, —C(Rₐ)═C(R_{b})—R_{c} or >C=C(Rₐ)—R_{b}, with Rₐ, R_{b}, and R_{c}, which may be identical or different, representing a hydrogen atom or a (C₁-C₄)alkyl group such as methyl, preferably hydrogen. In particular, the ethylenically unsaturated anhydride compound is a cyclic compound, which is preferably 5- or 6-membered, and comprising an ethylenic unsaturation.

As used herein, the term “hydrocarbon-based oil” means an oil formed essentially from, or even constituted of, carbon and hydrogen atoms, and optionally oxygen and nitrogen atoms, and not containing any silicon or fluorine atoms. It may contain hydroxy, ester, ether, carboxylic acid, amine and/or amide groups.

As used herein, the terms “include”, “includes” and “including” are meant to be non-limiting.

As used herein, the term “non polymeric compound” refers to a compound which is not directly obtained via a monomer polymerization reaction.

As used herein, the term “oil” means a fatty substance that is liquid at room temperature (25° C.) and at atmospheric pressure.

As used herein, the term “nonvolatile oil” refers to an oil with a vapor pressure of less than 0.13 Pa.

As used herein, the term “silicone oil” means an oil comprising at least one silicon atom and notably at least one Si—O group.

As used herein, the term “volatile oil” refers to an oil (or non-aqueous medium) that can evaporate on contact with the skin in less than one hour, at room temperature and at atmospheric pressure. The volatile oil is a volatile cosmetic oil, which is liquid at room temperature, notably having a nonzero vapor pressure, at room temperature and at atmospheric pressure, in particular having a vapor pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

A first aspect of the present disclosure is drawn to the two compositions of a two-step reactive lip system, a base coat and a top coat. The base coat is first applied to the lips, then the top coat is applied onto the base coat.

Embodiments of the disclosed base coat must contain a dispersion of copolymer particles surface-stabilized with one or more stabilizers in a non-aqueous medium containing at least one hydrocarbon-based oil, where the copolymer is a C₁-C₄ alkyl (meth)acrylate and maleic anhydride. Embodiments of the disclosed top coat must contain a silicone amine and a viscous silicone, the viscous silicone has a viscosity of at least about 100,000 centistoke when measured at a concentration by weight of 100% and using a rotational viscometer at 25° C. Each of these three elements will be described in additional detail below.

Dispersion of Copolymer Particles Surface-Stabilized with One or More Stabilizers

The dispersion of surface-stabilized copolymer particles comprises at least three components: (I) particles of at least one copolymer surface-stabilized with (II) at least one stabilizer in a preferably anhydrous medium, further containing (III) at least one hydrocarbon-based oil. Moreover, the disclosed dispersions contain particles, which are generally spherical, of at least one surface-stabilized polymer, in a non-aqueous medium.

The dispersion may be present in the base coat in an amount between about 10% and about 70% by weight in the base coat composition, and preferably between about 30% and about 50% by weight.

(I) Copolymer Particles

The particle(s) of the dispersion are constituted by one or more ethylenic copolymer(s) of (IA) (C₁-C₄)alkyl (C₁-C₄)(alkyl)acrylate and of (IB) an ethylenically unsaturated anhydride compound.

According to a preferred embodiment, the copolymer constituting the copolymer particles is a copolymer of acrylate:

(IA) of formula H₂C═C(R)—C(O)—O—R′ with R representing a hydrogen atom or a (C₁-C₄)alkyl group such as methyl, and R′ representing a (C₁-C₄)alkyl group such as methyl or ethyl, preferably C₁-C₄ alkyl (meth)acrylate; and

(TB) of an ethylenically unsaturated anhydride monomer.

Preferably, the copolymer particles are a polymer of C₁-C₄ alkyl (meth)acrylate and of ethylenically unsaturated anhydride monomer. More preferably, the copolymer particles are a copolymer of C₁-C₄ alkyl (meth)acrylate and maleic anhydride.

The monomers (IA) are preferably chosen from methyl (meth)acrylate, ethyl (meth)acrylate, n-propyl (meth)acrylate, isopropyl (meth)acrylate, n-butyl (meth)acrylate and tert-butyl (meth)acrylate.

A C₁-C₄ alkyl acrylate monomer is advantageously used. Preferentially, (IA) is chosen from methyl acrylate and ethyl acrylate.

The polymer of the particles also comprises an ethylenically unsaturated anhydride monomer b).

Preferentially, the ethylenically unsaturated anhydride compound(s) (IB) of are chosen from derivatives of maleic anhydride (IB1) and itaconic anhydride (IB2):

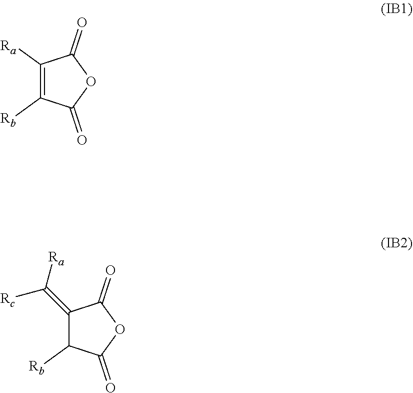

in which formulae (IB1) and (IB2) Rₐ, R_{b} and R_{c}, which may be identical or different, represent a hydrogen atom or a (C₁-C₄)alkyl group; preferably, Rₐ, R_{b}, and R_{c} represent a hydrogen atom.

More preferentially, the ethylenically unsaturated anhydride monomer is of formula (Ib) and even more preferentially is maleic anhydride.

According to a preferred embodiment, the polymer(s) of the particles comprise, or essentially consist of, from 80 to 99.99% by weight of monomer (IA) and from 0.01 to 20% by weight of monomer (IB), relative to the total weight of the polymer.

In certain embodiments, the copolymer of the particles may be: a methyl acrylate/maleic anhydride copolymer, an ethyl acrylate/maleic anhydride copolymer, or a methyl acrylate/ethyl acrylate/maleic anhydride copolymer.

Advantageously, the copolymer particles are comprised of a non-crosslinked polymer.

The copolymer particles preferably have a number-average molecular weight ranging from 2000 to 10 000 000.

The copolymer particles preferably have a number-average size ranging from 5 to 500 nm, notably ranging from 10 to 400 nm and better still ranging from 20 to 300 nm.

The copolymer particles may be present in the dispersion in an amount ranging from 20% to 60% by weight relative to the total weight of the dispersion of surface-stabilized copolymer particles, in particular between 21% and 58.5% by weight relative to the total weight of the dispersion of surface-stabilized copolymer particles, preferably ranging from 30% to 50% by weight relative to the total weight of the dispersion of surface-stabilized copolymer particles, and more preferentially ranging from 36% to 42% by weight relative to the total weight of the dispersion of surface-stabilized copolymer particles.

The copolymer particles may be present in the base coat in an amount between about 2% and about 42% by weight in the base coat composition, and preferably between about 6% and about 30% by weight, and still more preferably between about 10% and about 21% by weight.

The C₁-C₄ alkyl (meth)acrylate may be present in the base coat in an amount between about 1.6% and about 42% by weight in the base coat composition, and preferably between about 4.8% and about 30% by weight, and still more preferably between about 8% and about 21% by weight.

The maleic anhydride may be present in the base coat in an amount between about 0.02% and about 8.4% by weight in the base coat composition, and preferably between about 0.06% and about 6% by weight, and still more preferably between about 0.1% and about 4.2% by weight.

(II) One or More Stabilizers

One or more stabilizers may be used. Preferably, a single type of stabilizer is used.

The stabilizers are constituted of ethylenic polymers chosen from (IIC) polymers of (C₃-C₁₂)cycloalkyl (C₁-C₆)(alkyl)acrylate monomers; and (IID) copolymers of (C₃-C₁₂)cycloalkyl (C₁-C₆)(alkyl)acrylate and (C₁-C₄)alkyl (C₁-C₄)(alkyl)acrylate.

According to a preferred embodiment of the invention, the stabilizers are constituted of ethylenic polymers chosen from

(IIC) polymers of monomers of formula H₂C═C(R)—C(O)—O—R″ with R representing a hydrogen atom or (C₁-C₄)alkyl group such as methyl, and R″ representing a (C₅-C₁₀)cycloalkyl group such as norbornyl or isobornyl, preferably isobornyl; and

(IID) copolymers of H₂C═C(R)—C(O)—O—R′ and of H₂C═C(R)—C(O)—O—R″ with R, R′ and R″ as defined previously.

In preferred embodiments, the stabilizer is an isobornyl (meth)acrylate polymer chosen from: (i) isobornyl (meth)acrylate homopolymer and/or (ii) statistical copolymers of isobornyl (meth)acrylate and C₁-C₄ alkyl (meth)acrylate which are preferably present in an isobornyl (meth)acrylate/C₁-C₄ alkyl (meth)acrylate weight ratio of greater than 4. Advantageously, this weight ratio ranges from 4.5 to 19. For the statistical copolymers, the defined weight ratio makes it possible to obtain a polymer dispersion that is stable, notably after storage for seven days at room temperature.

In preferred embodiments, the stabilizer is: (i) an isobornyl acrylate homopolymer; (ii) a statistical copolymer of isobornyl acrylate/methyl acrylate; (iii) a statistical copolymer of isobornyl acrylate/ethyl acrylate; or (iv) a statistical copolymer of isobornyl acrylate/methyl acrylate/ethyl acrylate according to the weight ratio described previously.

Advantageously, the sum of stabilizer(s)+copolymer particles comprises from 10% to 50% by weight of copolymers (IID) and from 50 to 90% by weight of polymers (IIC), relative to the total weight of the sum of stabilizer(s)+copolymer particles.

Preferentially, the sum of stabilizer(s)+copolymer particle(s) present in the dispersion comprise from 15% to 30% by weight of copolymers (IID) and from 70% to 85% by weight of polymers (IIC), relative to the total weight of the sum of stabilizer(s)+copolymer particle(s).

In some embodiments, the surface-stabilized copolymer particles comprise between about 10% and about 30% by weight of the one or more stabilizers, between about 50% and about 89.99% by weight of the C₁-C₄ alkyl (meth)acrylate, and between about 0.01% and about 25% of maleic anhydride.

The stabilizer may be present in the base coat in an amount between about 0.2% and about 18% by weight in the base coat composition, and preferably between about 0.6% and about 13% by weight, and still more preferably between about 1% and about 9% by weight.

(III) Hydrocarbon-Based Oil

The dispersion comprises one or more identical or different, preferably identical, hydrocarbon-based oils. The hydrocarbon-based oil may be volatile or nonvolatile. According to a preferred embodiment, the hydrocarbon-based oil(s) are volatile or are a mixture of different volatile oils, more preferentially chosen from isododecane and octyldodecanol. According to another particular embodiment, the hydrocarbon-based oil(s) are a mixture of a volatile oil and a nonvolatile oil.

The hydrocarbon-based oil may optionally be chosen from:

(i) hydrocarbon-based oils containing from 8 to 14 carbon atoms, and notably branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl;

(ii) linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155 059 from the company Cognis, and mixtures thereof;

(iii) short-chain esters (containing from 3 to 8 carbon atoms in total) such as ethyl acetate, methyl acetate, propyl acetate or n-butyl acetate;

(iv) hydrocarbon-based oils of plant origin such as triglycerides constituted of fatty acid esters of glycerol, the fatty acids of which may have chain lengths varying from C4 to C24, these chains possibly being linear or branched, and saturated or unsaturated; these oils are notably heptanoic or octanoic acid triglycerides, or alternatively wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame oil, marrow oil, rapeseed oil, blackcurrant oil, evening primrose oil, millet oil, barley oil, quinoa oil, rye oil, safflower oil, candlenut oil, passion-flower oil and musk rose oil; shea butter; or else caprylic/capric acid triglycerides, for instance those sold by the company Stéarineries Dubois or those sold under the names Miglyol 810®, 812® and 818® by the company Dynamit Nobel;

(v) synthetic ethers containing from 10 to 40 carbon atoms;

(vi) linear or branched hydrocarbons of mineral or synthetic origin, such as petroleum jelly, polydecenes, hydrogenated polyisobutene such as Parleam®, squalane and liquid paraffins, and mixtures thereof;

(vii) synthetic esters such as oils of formula R₁C(O)—O—R₂ in which R₁ represents a linear or branched fatty acid residue including from 1 to 40 carbon atoms and R₂ represents an, in particular branched, hydrocarbon-based chain containing from 1 to 40 carbon atoms, on the condition that R₁+R₂≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters; and/or

(viii) fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

The dispersion may also comprise a silicone oil, in addition to the hydrocarbon-based oil. If a silicone oil is in the dispersion, it is preferably in an amount which does not exceed 10% by weight relative to the weight of the dispersion, more particularly in an amount of less than 5% and more preferentially 2%. The silicone oil may be volatile or nonvolatile.

Volatile silicone oils may include, but are not limited to, volatile linear or cyclic silicone oils, notably those with a viscosity 8 centistokes (cSt) (8×10⁻⁶ m²/s), and notably containing from 2 to 10 silicon atoms and in particular from 2 to 7 silicon atoms, these silicones optionally including alkyl or alkoxy groups containing from 1 to 10 carbon atoms. Certain embodiments utilize dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Nonvolatile silicone oils may include, but are not limited to, linear or cyclic nonvolatile polydimethylsiloxanes (PDMSs); polydimethylsiloxanes including alkyl, alkoxy and/or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates and pentaphenyl silicone oils.

In certain embodiments, the dispersion comprises a hydrocarbon-based oil in an amount ranging from 60 to 100% by weight of the total weight of the oils present in the composition, and silicon oil in an amount ranging from 0 to 40% by weight of the total weight of the oils present in the composition. According to a preferred embodiment of the invention, the composition contains as oil only a hydrocarbon-based oil.

Advantageously, the hydrocarbon-based oils of the invention are apolar, i.e., formed solely of carbon and hydrogen atoms.

In some embodiments, the hydrocarbon-based oils are preferably chosen from hydrocarbon-based oils containing from 8 to 14 carbon atoms, which are in particular volatile, more particularly the apolar oils, described previously.

In some embodiments, The hydrocarbon-based oils are preferably chosen from hydrocarbon-based oils containing from 10 to 18 carbon atoms, which are in particular volatile, more particularly the apolar oils, described previously.

Preferentially, the hydrocarbon-based oil(s) of the invention are isododecane.

According to another embodiment, the hydrocarbon-based oil(s) are a mixture of nonvolatile and volatile oil; preferably, the mixture comprises isododecane as volatile oil. In particular, in the mixture, the nonvolatile oil is a phenyl silicone oil, preferably chosen from pentaphenyl silicone oils.

Method for Preparing the Dispersion

A non-limiting example of preparing the dispersion is as follows. The polymerization is performed in “dispersion”, i.e., by precipitation of the polymer being formed, with protection of the particles formed with one or more stabilizers, preferably one stabilizer.

In a first step, the stabilizers are prepared by mixing the constituent monomer(s) of the stabilizing polymers (IIC) or (IID) (discussed above) with a free-radical initiator, in a solvent known as the synthesis solvent, and by polymerizing these monomers.

In a second step, the constituent monomers of the copolymer particles are added to the stabilizing polymer formed in the preceding step and polymerization of these added monomers is performed in the presence of the free-radical initiator.

When the nonaqueous medium is a nonvolatile hydrocarbon-based oil, the polymerization may be performed in an apolar organic solvent (synthesis solvent), followed by adding the nonvolatile hydrocarbon-based oil (which should be miscible with said synthesis solvent) and selectively distilling off the synthesis solvent.

Dye(s) and/or pigment(s) may be added during the first step. According to another variant, the dye(s) and/or pigment(s) are added during the second step or after the second step.

A synthesis solvent which is such that the monomers of the stabilizing polymer and the free-radical initiator are soluble therein, and the polymer particles obtained are insoluble therein, so that they precipitate therein during their formation, is thus chosen.

In particular, the synthesis solvent is chosen which is apolar and organic, preferably chosen from alkanes such as heptane or cyclohexane.

When the nonaqueous medium is a volatile hydrocarbon-based oil, the polymerization may be performed directly in said oil, which thus also acts as synthesis solvent. The monomers should also be soluble therein, as should the free-radical initiator, and the copolymer particles which are obtained should be insoluble therein.

The monomers are preferably present in the synthesis solvent, before polymerization, in a proportion of 5% to 45% by weight. The total amount of the monomers may be present in the solvent before the start of the reaction, or part of the monomers may be added gradually as the polymerization reaction proceeds.

The polymerization is preferentially performed in the presence of one or more free-radical initiators, notably of the type such as:

(i) peroxide, in particular chosen from tert-butyl peroxy-2-ethylhexanoate: Trigonox 21S; 2,5-dimethyl-2,5-bi s(2-ethylhexanoylperoxy)hexane: Trigonox 141; tert-butyl peroxypivalate: Trigonox 25C75 from AkzoNobel; or

(ii) azo, in particular chosen from AIBN: azobisisobutyronitrile; V50: 2,2′-azobis(2-amidinopropane) dihydrochloride.

The polymerization is preferably performed at a temperature ranging from 70° C. to 110° C. and at atmospheric pressure.

The copolymer particles are surface-stabilized, when they are formed during the polymerization, by means of the stabilizer. The stabilization may be performed by any known means, and in particular by direct addition of the stabilizer, during the polymerization. The stabilizer is preferably also present in the mixture before polymerization of the monomers of the copolymer. However, it is also possible to add it continuously, notably when the monomers of the copolymer are also added continuously. From 10% to 30% by weight and preferably from 15% to 25% by weight of the stabilizer(s) may be used relative to the total weight of monomers used (stabilizers (II))+copolymer particles (I)).

The dispersion advantageously comprises from 30% to 65% by weight of solids relative to the total weight of said dispersion and preferably from 40% to 60% by weight relative to the total weight of said dispersion.

The dispersion may comprise an amount of copolymer particles (I)+stabilizers (II) ranges from 1% to 50% by weight, preferably ranging from 2% to 30% by weight, relative to the total weight of the dispersion.

According to a preferred embodiment of the invention, the dispersion according to the invention is an anhydrous composition.

According to another embodiment of the present patent application, the dispersion is an inverse emulsion, i.e., of water-in-oil type (W/O). In this case, the composition comprises one or more surfactants, which are preferably nonionic.

Silicone Amine

The top coat should comprise a silicone amine. In preferred embodiments, the silicone amine is present in an amount of between 0.5% and 40% by weight in the top coat composition.

In some embodiments, the silicone amine compounds used in the process of the invention are preferably chosen from amino alkoxysilanes, such as those of formula (IV1):

R′₁—Si(OR′₂)_{z}(R′₃)ₓ (IV1)

where R′₁ is a linear or branched, saturated or unsaturated, cyclic or acyclic C₁-C₁₀ hydrocarbon-based chain substituted with one or more groups chosen from the groups:

(i) primary amine NH₂ or secondary amine N(H)R with R representing a (C₁-C₄)alkyl group,

(ii) aryl or aryloxy substituted with an amino or (C₁-C₄)alkylamino group or with a C₁-C₄ aminoalkyl group, and

(iii) aldehyde —C(O)—H, carboxy —C(O)—OH, amide —C(O)—NH₂ or urea-NH—C(O)—NH₂; R′₁ is optionally interrupted in its hydrocarbon-based chain with one or more heteroatoms (notably O, S, NH), a carbonyl group (CO), or the combination thereof, such as ester —C(O)—O—, or amide —C(O)—NH—, R′1 being bonded directly to the silicon atom via a carbon atom,

R′₂ and R′₃ which may be identical or different, represent a linear or branched alkyl group comprising from 1 to 6 carbon atoms, z denotes an integer ranging from 1 to 3, and x denotes an integer ranging from 0 to 2, with z+x=3.

Preferably, R′₂ represents an alkyl group comprising from 1 to 4 carbon atoms.

Preferably, R′₂ represents a linear alkyl group, comprising from 1 to 4 carbon atoms.

Preferably, R′₂ represents an ethyl group.

Preferably, R′₃ represents an alkyl group comprising from 1 to 4 carbon atoms.

Preferably, R′₃ represents a linear alkyl group, comprising from 1 to 4 carbon atoms.

Preferably, R′₃ represents a methyl or ethyl group.

Preferably, R′₁ is an acyclic chain.

Preferably, R′₁ is a linear or branched, saturated or unsaturated C₁-C₆ hydrocarbon-based chain substituted with an amine NH₂ or N(H)R group, with R representing a C₁-C₆ alkyl, C₃-C₆ cycloalkyl, or C₆ aromatic group.

Preferentially, R′₁ is a saturated linear C₁-C₆ hydrocarbon-based chain substituted with an amine group NH₂.

More preferentially, R′₁ is a saturated linear C₂-C₄ hydrocarbon-based chain substituted with an amine group NH₂.

Preferably, R′₁ is a saturated linear C₁-C₆ hydrocarbon-based chain substituted with an amine group NH₂.

R′₂ represents an alkyl group comprising from 1 to 4 carbon atoms,

R′₃ represents an alkyl group comprising from 1 to 4 carbon atoms.

Preferably, z is equal to 3.

Preferably, the amino alkoxysilane of formula (IV1) is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

More preferably, the amino alkoxysilane (IV1) is chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane.

Still more preferably, the amino alkoxysilane (IV1) is 3-aminopropyltriethoxysilane (APTES).

The silicone polymers may also be polydi(C₁-C₄)alkylsiloxanes, in particular polydimethylsiloxanes, comprising amine groups at the chain end or on side chains are particularly end or side amino(C₁-C₆)alkyl groups such as aminopropyl, more particularly those of formula (IV2) or (IV3) or (IV4):

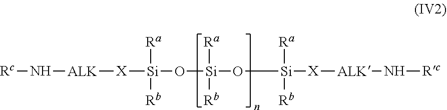

where R^{a} and R^{b}, which may be identical or different, preferably identical, represent a (C₁-C₄)alkyl group such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl, R^{c} and R′^{c}, which may be identical or different, preferably identical, represent a hydrogen atom, a (C₁-C₄)alkyl group, an amino(C₁-C₄)alkyl or (C₁-C₄)alkylamino(C₁-C₄)alkyl group, preferably a hydrogen atom or an amino(C₁-C₄)alkyl group such as aminoethyl; X represents a covalent bond, an oxygen atom, preferably a covalent bond; ALK and ALK′, which may be identical or different, preferably identical, represent a (C₁-C₆)alkylene group, preferably (C₁-C₄)alkylene such as propylene; n representing an integer greater than 2 and more particularly the value of n is such that the weight-average molecular weight of the silicone is between 500 and 55 000;

preferentially, the polydi(C₁-C₄)alkylsiloxanes of formula (IV2) are of formula (IV′2) or (IV′″2) below:

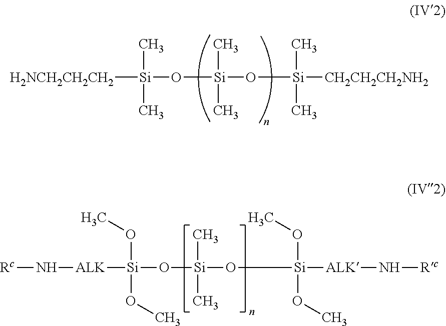

in which formula (IV2) the value of n is such that the weight-average molecular weight of the silicone is between 500 and 55 000. As an example of aminosilicone (IV2) or (IV′2), mention may be made of those sold under the names DMS-A11, DMS-A12, DMS-A15, DMS-A21, DMS-A31, DMS-A32 and DMS-A35 by the company Gelest. For formula (IV″2), R^{c}, R′^{c}, ALK, ALK′, and n as defined previously for (IVb). Preferably, ALK and ALK′ are identical and represent a (C1-C4)alkylene group such as propylene, R^{c} and R′^{c} are identical and represent an amino(C1-C4)alkyl group such as aminoethyl;

in particular, Dimethoxysilyl Ethylenediaminopropyl Dimethicone (RN: 71750-80-6), under the trade name GP-RA-157, sold by Genesee Polymers may be utilized;

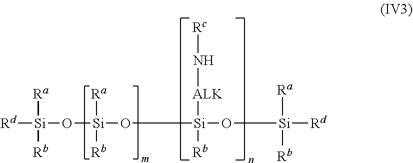

where R^{a}, R^{b}, and R^{d}, which may be identical or different, preferably identical, represent a (C₁-C₄)alkyl group such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl, R^{d} may also represent a (C₁-C₆)alkyl group substituted with a (C₁-C₄)alkylamino or amino group, R^{c} represents a hydrogen atom or a (C₁-C₄)alkyl group, preferably a hydrogen atom; ALK represents a (C₁-C₆)alkyl group, preferably (C₁-C₄)alkylene such as propylene; n and m, which may be identical or different, represent an integer greater than 2 and more particularly the values of m and n are such that the weight-average molecular weight of the silicone is between 1000 and 55 000. Preferentially, the polydi(C1-C4)alkylsiloxanes of formula (IV3) have the formula (IV′3) below:

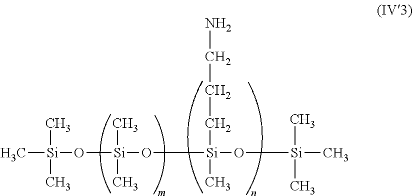

where the values of n and m are such that the weight-average molecular weight of the silicone is between 1000 and 55 000. As examples of silicone (IV3), mention may be made of those sold under the names AMS-132, AMS-152, AMS-162, AMS-163, AMS-191 and AMS-1203 by the company Gelest;

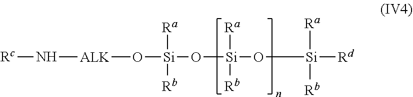

where R^{a} and R^{b}, which may be identical or different, preferably identical, represent a (C₁-C₄)alkyl group such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl, and R^{d} represents a (C₁-C₆)alkyl group optionally substituted with a (C₁-C₄)alkylamino or amino group, preferably (C₁-C₄)alkyl, such as isobutyl, tert-butyl or n-butyl, R^{c} represents a hydrogen atom or a (C₁-C₄)alkyl group, preferably a hydrogen atom; ALK represents a (C₁-C₆)alkylene group, preferably (C₁-C₄)alkylene such as propylene, n representing an integer greater than 2 and more particularly the value of n is such that the weight-average molecular weight of the silicone is between 500 and 5000; and preferably, the polydi(C₁-C₄)alkylsiloxanes of formula (IV4) have the formula (IV′4) below:

H₂NCH₂CH₂CH₂—Si(CH₃)₂—O—[Si(CH₃)₂—O]n—Si(CH₃)₂C₄H₉ (IV′4)

where the value of n is such that the weight-average molecular weight of the silicone is between 500 and 3000. As examples of silicones (IV4), mention may be made of the products sold under the names MCR-A11 and MCR-A12 by the company Gelest.

The silicone amine may also include amodimethicones of formula (IV5):

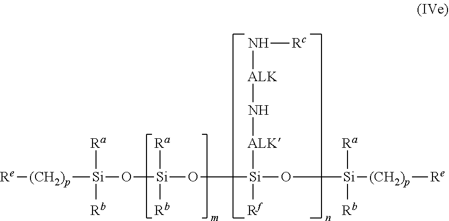

where R^{a} and R^{b}, which may be identical or different, preferably identical, represent a (C₁-C₄)alkyl group such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl,

R^{c} represents a hydrogen atom or a (C₁-C₄)alkyl group, preferably a hydrogen atom;

R^{e} represents a hydroxyl, (C₁-C₄)alkoxy, amino or (C₁-C₄)alkylamino group,

R^{f} represents a (C₁-C₄)alkyl group such as methyl, (C₁-C₄)alkoxy such as methoxy, a hydroxyl group or —O—(SiR₂)ₓ—R′ with R representing a (C₁-C₄)alkyl or (C₁-C₄)alkoxy group and R′ representing a (C₁-C₄)alkoxy or hydroxyl group; preferably, R^{f} represents a (C₁-C₄)alkyl, (C₁-C₄)alkoxy or —O—(SiR₂)ₓ—R′ group with R representing a (C₁-C₄)alkyl group such as methyl and R′ a hydroxyl or (C₁-C₄)alkoxy group such as methoxy;

R^{g} represents a hydrogen atom or a (C₁-C₆)alkyl group

ALK and ALK′, which may be identical or different, represent a (C₁-C₆)alkylene group, preferably (C₁-C₄)alkylene such as ethylene or propylene; n and m, which may be identical or different, represent an integer greater than 2, p and x are integers greater than or equal to 0; preferably, p is between 2 and 20 and more particularly the values of m, n, p and x are such that the weight-average molecular weight of the silicone is between 2000 and 700 000, preferentially between 5000 and 500 000; and preferentially, the amodimethicones of formula (IV5) are of formula (IV′5) or (IV′″5) below:

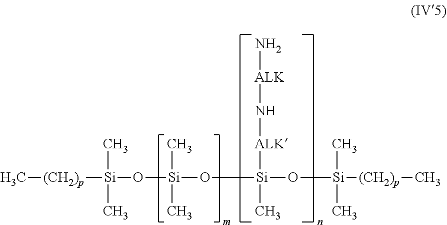

where ALK represents a (C₁-C₆)alkylene group, preferably ethylene, ALK′ represents a (C₁-C₆)alkylene group, preferably propylene, and m, n and p which may be identical or different, represent an integer greater than 2, with m, n and p such that the weight-average molecular weight of the compound is between approximately 5000 and 500 000; preferably, p represents an integer of between 8 and 20;

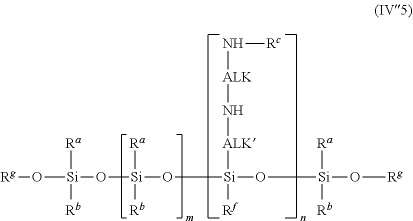

where R^{a} and R^{b}, which may be identical or different, preferably identical, represent a (C₁-C₄)alkyl group such as methyl, (C₁-C₄)alkoxy such as methoxy, preferably (C₁-C₄)alkyl such as methyl, R^{c} represents a hydrogen atom or a (C₁-C₄)alkyl group, preferably a hydrogen atom; R^{f} represents a (C₁-C₄)alkyl group such as methyl, (C₁-C₄)alkoxy such as methoxy, or —O—(SiR₂)ₓ—R′ with R representing a (C₁-C₄)alkyl group such as methyl and R′ a hydroxyl or (C₁-C₄)alkoxy group such as methoxy; R^{g} represents a hydrogen atom or a (C₁-C₆)alkyl group, ALK represents a (C₁-C₆)alkylene group, preferably ethylene, ALK′ represents a (C₁-C₆)alkylene group, preferably propylene, n and m, which may be identical or different, representing an integer greater than 2, x is an integer greater than or equal to 0; preferably, the values of m, n and x are such that the weight-average molecular weight of the silicone is between 2000 and 700 000, preferentially between 5000 and 500 000;

Even more particularly, (IV″5) is represented by the formula (IV′″5)

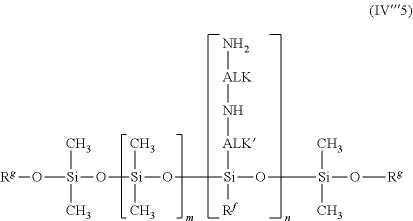

where R^{f}, R^{g}, ALK, ALK′, m, n as defined for (IV″5). The amodimethicones and trimethylsiloxy amodimethicones belonging to formula (IV″5) and the formula above are, for example, the amodimethicones and trimethylsiloxy amodimethicones of ADM type sold by the company Wacker-Belsil®.

Mention may also be made of polydimethylsiloxanes bearing aminoethylaminopropyl groups, bearing a methoxy and/or hydroxyl function and α-ω silanols as a cationic 60% aqueous emulsion (supplier reference: Xiameter MEM-8299 Emulsion by Dow Corning or supplier reference: Belsil ADM 4000 E by Wacker).

The silicone amine may be a polydimethylsiloxane bearing an aminoethyl iminopropyl group, as a stored nonionic 15% microemulsion (supplier reference: Belsil ADM Log 1).

Preferred embodiments utilize polydi(C₁-C₄)alkylsiloxanes comprising primary amine groups at the chain end and/or on side chains.

In preferred embodiments, the silicone amine comprises diamines or triamines. When the top coat is applied, the diamines or triamines can then react with different maleic anhydride groups, allowing a fast crosslinking reaction to occur at normal temperatures for applying lipsticks (e.g., around room temperature, or around body temperature).

Viscous Silicone

The viscous silicone has a viscosity of at least about 100,000 centistoke (cSt) when measured at a concentration by weight of 100% and using a rotational viscometer at 25° C.

Procedure

Viscosity may be measured using a rotational viscometer such as a Brookfield Synchro-lectric viscometer or a Wells-Brookfield Cone/Plate viscometer using a method similar to ASTM D 1084 Method B or ASTM D 4287 (for cone/plate). Temperature is maintained at 25 C and a suitable viscometer such as Brookfield Synchro-lectric viscometer or Wells-Brookfield Cone/Plate viscometer, Brookfield Engineering Laboratories, Stoughton, Mass. is used.

A container of sufficient diameter to avoid wall effects is filled with sample to a level such that the spindle can be immersed, and air is not trapped in the sample. Spindle and speed are selected. The shaft should be centered in the bottle and the fluid level should be at the mark on the shaft. Level the viscometer. Depress the clutch and start the motor. Release the clutch and allow the dial to rotate until the pointer stabilizes at a fixed point in the dial unless otherwise specified. Using the dial reading and the factor given for the model, spindle and speed used, calculate the viscosity.

If indicated by the manufacturer, to calculate viscosity from “dial” readings on the viscometer, (e.g., % torque), multiply by factor indicated based on spindle size and rotation speed: Viscosity (cP)=(Dial reading)*(Factor). To convert to centistokes, viscosity (cSt)=Viscosity (cP)/density of the sample (g/cm³).

The viscous silicone may be present in a total amount of between about 0.1% and about 50% by weight in the top coat composition, and preferably in an amount between 5% and 30% by weight.

Viscous silicones may include, but are not limited to, silicone gums (INCI name: Dimethiconol) alone or as a mixture with a silicone oil, such as the product marketed by the company Dow Corning under the name Dow Corning 1501 Fluid, which is a mixture of Dimethiconol and Cyclopentasiloxane in the ratio 14.7/85.3 (dimethiconol); silicone elastomers (including silicone thermoplastic elastomers) such as those marketed under the names KSG by the company Shin-Etsu; silicone waxes; and combinations thereof.

In one embodiment, the viscous silicones includes co-emulsifiers, such as, but not limited to, lauryl PEG-9 polydimethylsiloxyethyl dimethicone, PEG-10 dimethicone, PEG-9 polydimethylsiloxyethyl dimethicone (and) PEG-9. The co-emulsifiers facilitate stability of the composition. In another embodiment, the oil phase may also include cosmetically acceptable additives or adjuvants as well as cosmetic or dermatologic active agents.

The base coat and/or top coat may also contain other ingredients, such as colorants, additional hydrocarbon-based oils, additional silicone oils, a styrenic block copolymer, etc.

In some embodiments, the basecoat and/or topcoat may contain between 0% and 50% by weight of additional hydrocarbon-based oils or silicone oils in a given composition (base coat and/or top coat). In some embodiments, the basecoat and/or topcoat may preferably contain no additional hydrocarbon-based oils or silicone oils. In some embodiments, the basecoat and/or topcoat may preferably contain between 20% and 30% by weight of additional hydrocarbon-based oils or silicone oils. In some embodiments, the additional hydrocarbon-based oil is isododecane.

Colorants

According to certain embodiments of the present application, the base coat may also utilize at least one colorant. In certain embodiments, the colorant is a pigment, a pearlescent agent, or a combination thereof. The combined colorants should be present in the base coat in a total amount less than or equal to 40% by weight in the base coat, preferably less than or equal to 30% by weight, more preferably less than or equal to 20% by weight, and still more preferably less than or equal to 15% by weight, and most preferably less than or equal to 10% by weight. In certain embodiments, the combined colorants are be present in the base coat in a total amount greater than or equal to 0.001% by weight, and preferably greater than or equal to 0.5% by weight. In preferred embodiments, the combined colorants are be present in the base coat in a total amount between 0.5% and 40% by weight. In other embodiments, the combined colorants are present in the base coat in a total amount between 0.001% and 40% be weight. In still other embodiments, the combined colorants are present in the base coat in a total amount between 1% and 30%. In some embodiments, the combined colorants are present in the base coat in a total amount between 5% and 30%. In some embodiments, one or more of the colorants are pigments.

In preferred embodiments, the second composition contains no colorant.

Suitable colorants include, but are not limited to, lipophilic dyes, pigments and pearlescent agents, and their mixtures. Any colorant typically found in lipstick compositions can be used.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescents may also be included, and may be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Color additives, such as natural extracts, may also be appropriate in various embodiments. One such example is spirulina paltensis extract, although other extracts may also be appropriate.

In preferred embodiments, the colorant(s) is/are present in a total amount of between 0.5% and 40% by weight in the base coat composition. In more preferred embodiments, colorant(s) is/are present in a total amount of between 1% and 30% by weight. In still more preferred embodiments, the colorant(s) is/are present in a total amount of between 5% and 30% by weight. In some embodiments, one or more of the colorants are pigments.

Styrenic Block Copolymer

The first component in the base coat is at least one styrenic block copolymer.

Suitable examples of styrenic block copolymers include the class of Kraton™ rubbers (Shell Chemical Company) or from similar thermoplastic elastomers. Kraton™ rubbers are thermoplastic elastomers in which the polymer chains comprise a di-block, tri-block, multi-block or radial or star block configuration or numerous mixtures thereof. The Kraton™ tri-block rubbers have polystyrene (hard) segments on each end of a rubber (soft) segment, while the Kraton™ di-block rubbers have a polystyrene (hard) segment attached to a rubber (soft) segment. The Kraton™ radial or star configuration may be a four-point or other multipoint star made of rubber with a polystyrene segment attached to each end of a rubber segment. The configuration of each of the Kraton™ rubbers forms separate polystyrene and rubber domains.

Each molecule of Kraton™ rubber is said to comprise block segments of styrene monomer units and rubber monomer and/or co-monomer units. The most common structure for the Kraton™ triblock copolymer is the linear A-B-A block type styrene-butadiene-styrene, styrene-isoprene-styrene, styrene-ethylene/propylene-styrene, or styrene-ethylene/butylene-styrene.

The Kraton™ di-block is preferably the AB block type such as styrene-ethylene/propylene, styrene-ethylene/butylene, styrene-butadiene, or styrene-isoprene. The Kraton™ rubber configuration is well known in the art and any block copolymer elastomer with a similar configuration is within the practice of the disclosed. Other block copolymers are sold under the tradename Septon (which represent elastomers known as SEEPS, sold by Kurary, Co., Ltd) and those sold by Exxon Dow under the tradename Vector™.

Other thermoplastic elastomers useful in the present invention include those block copolymer elastomers comprising a styrene-butylene/ethylene-styrene copolymer (tri-block) (sometimes referred to as a styrene and ethylene/butylene triblock copolymer), an ethylene/propylene-styrene copolymer (radial or star block) or a mixture or blend of the two. (Some manufacturers refer to block copolymers as hydrogenated block copolymers, e.g., hydrogenated styrene-butylene/ethylene-styrene copolymer (tri-block)). Hydrogenated styrene/butadiene copolymer (Kraton G1657), commercially available from Kraton Polymers, may also be used.

Preferably, the at least one styrenic block copolymer is present in an amount ranging from about 1% to about 20% by weight of active material with respect to the total weight of the base coat, preferably from about 5% to about 20%, more preferably from about 7% to about 20%, and more preferably from about 9 to about 20% by weight with respect to the total weight of the base coat composition. In some embodiments, the at least one styrenic block copolymer is present in an amount ranging from about 2% to about 15% by weight of active material with respect to the total weight of the base coat composition.

The styrenic block copolymer may be present in the base coat in an amount between about 1% and about 20% by weight in the base coat composition.

### Example #1

Referring to the compositions listed in Tables 1 and 2, all ingredients were combined and mixed at room temperature until homogenous. The base coat and top coat were then transferred into a desired container.

TABLE 1
Disclosed Base Coat Formulations

| | Form. 1 | Form. 2 |
|---|---|---|
| Material | % w/w | % w/w |
| Copolymer Dispersion | 15-25% | 25-35% |
| (50% In Isododecane) | | |
| Isododecane | 30-70% | 30-70% |
| Colorants (Pigments) | 0.5-40% | 0.5-40% |

TABLE 2
Disclosed Top Coat Formulas

| | Form. 3 |
|---|---|
| Material | % w/w |
| Silicone Amine | 0.5-40% |
| Viscous Silicones | 0-99.5% |
| Isododecane | 0-99.5% |

Evaluations

The evaluation results of the combined base coat and top coats are summarized in Table 3. The formulations were evaluated based on their adhesion testing (resistance to ASTM tape) and solvent resistance (resistance to olive oil, acetic acid (vinegar), and artificial saliva), as compared to a standard commercial product.

Adhesion Testing. A film of a sample was applied onto a black byko-chart Black Scrub Panels P121-10N #5015 using a 1 mil draw down bar. The films were allowed to dry at 35° C. and 60% RH for 15-30 minutes, after which 3 mil draw down of a top coat formulation was applied to the base coat, and allowed to dry overnight at 35° C. at 60% RH. Adhesion testing was performed on one end of the drawdown. A piece of ASTM crosshatch adhesive tape (Intertape 51596) was placed on the sample and removed at a 180° angle. A rating scale was used to assess the degree of sample removal. For example, a rating scale such as a scale of 1-3 can be used to assess the degree of sample removal from the substrate onto the tape, in which 1 is essentially no removal (V. Good), 2 is some removal (Good), and 3 is essentially complete removal (Poor).

Solvent resistance. The unused portion of the samples used for adhesion testing, were then used to evaluate for solvent resistance. To evaluate the samples, two drops of olive oil, then two drops of artificial saliva, and two drops of 2% acetic Acid was placed on 6 different sections of the film. Let sit for 10 minutes. Fold one Kim Wipe into a square for each drop area. Gently wipe the drop 15 times. Observe how much product wipes off onto the wipe and how/if the product moves on the byko-chart substrate. Following the abrasion, the film and the paper towel were assessed for removal/loss of sample. The same rating scale described above was used to assess the degree of sample removal.

TABLE 3
Summary of Evaluations

| Evaluation | 1 + 3 | 2 + 3 |
|---|---|---|
| Resistance to Olive Oil | Good | Good |
| Resistance to Acetic Acid | V. Good | V. Good |
| Resistance to Artificial Saliva | V. Good | V. Good |
| Resistance to ASTM tape | V. Good | V. Good |

### Example #2

Referring to the compositions listed in Tables 4 and 5, all ingredients were combined and mixed at room temperature until homogenous. The base coat and top coat were then transferred into a desired container.

TABLE 4
Base Coat Formula

| | Base A |
|---|---|
| Material | % w/w |
| Copolymer Dispersion | 10-70% |
| (50% In Isododecane) | |
| Pigments | 1.5-50% |
| (30% in Isododecane) | |
| Isododecane | 10-50% |

TABLE 5
Top Coat Formulas

| | Top A | Top B | Top C |
|---|---|---|---|
| Material | % w/w | % w/w | % w/w |
| Silicone Amine | 0.5-40% | 0.5-40% | — |
| Viscous Silicone | — | 60-99.5% | 80-99% |
| (15% solid content in Isododecane) | | | |
| Isododecane | 60-99.5% | — | 1-20% |

Evaluations

The evaluation results of the base coat and top coats are summarized in Table 6. The formulations were evaluated based on their shine values, adhesion testing (resistance to ASTM tape) as described above, solvent resistance (resistance to olive oil, acetic acid (vinegar), and artificial saliva) as described above, shine, and shine retention, as compared to a standard commercial product.

Top coats containing the viscous silicone in conjunction with silicone amine show a surprising improvement of the wear of the top coat and the wear of shine. For example, the top coat containing the viscous silicone and silicone amine (Base A Top B) retaining its integrity and shine lastingness more so than the same sample made without the incorporation of silicone amine in the top coat (Base A Top C). When comparing Base A Top C vs Base A Top B, the wear in the presence of olive oil, artificial saliva, and acetic acid is maintained in both cases, but the shine values and long lastingness of shine is improved when silicone amine is incorporated in the top coat with the high molecular weight silicone.

Shine. The shine of the film is first analyzed using a gloss meter (BYK: micro-TRI-gloss) at an angle of 20° and 60°. The higher the value the shinier the film.

Shine Retention: Following the test for solvent resistance, the integrity of the film (especially the top coat) was visually assessed for shine retention. The top coat is visually assessed for removal upon wiping. Samples were assessed immediately post wiping and 24 hours later. For samples with poor shine retention, the shine was reduced and top coat appeared disrupted. Good Shine retention, showed no disruption of the film and equivalent shine profile to the remainder of the non-perturbed film.

TABLE 6
Summary of Evaluations

| | | Abrasion | | | | | |
|---|---|---|---|---|---|---|---|
| Base | Top | | | Acetic | | | Shine |
| Coat | Coat | Oil | Saliva | Acid | Adhesion | Shine | Retention |
| A | A | 1.125 | 1 | 1 | 1.1 | 8 | Top Coat |
| | | | | | | | Retained |
| A | B | 1 | 1 | 1 | 1.06 | 61.2 | Top Coat |
| | | | | | | | Retained |
| A | C | 1 | 1 | 1 | 1.04 | 47.2 | Top Coat |
| | | | | | | | Removed |

### Example #3

Referring to the compositions listed in Tables 4 and 5, all ingredients were combined and mixed at room temperature until homogenous. The base coat and top coat were then transferred into a desired container.

TABLE 7
Base Coat Formula

| | Base E |
|---|---|
| Material | % w/w |
| Copolymer Dispersion | 10-70% |
| (50% In Isododecane) | |
| Pigments | 10-40% |
| (30% in Isododecane) | |
| Styrenic Block Copolymer | 10-40% |
| (26.7% in Isododecane) | |

TABLE 8
Top Coat Formulas

| | Top B | Top C |
|---|---|---|
| Material | % w/w | % w/w |
| Silicone Amine | 0.5-40% | — |
| Viscous Silicone | 60-99.5% | 60-99.5% |
| (15% solid content in Isododecane) | | |
| Isododecane | — | 0.5-40% |

Evaluations

The evaluation results of the base coat and top coats are summarized in Table 9. The formulations were evaluated as described above based on their shine values, adhesion testing (resistance to ASTM tape), solvent resistance (resistance to olive oil, acetic acid (vinegar), and artificial saliva), shine, and shine retention.

As can be seen, especially in comparison with the evaluations in Table 6, the addition of a styrenic block copolymer significantly increases shine, without impacting shine retention. As discussed previously, the addition of the silicone amine also significantly increases shine, as well as improving shine retention.

TABLE 9
Summary of Evaluations

| | | Abrasion | | | | | |
|---|---|---|---|---|---|---|---|
| Base | Top | | | Acetic | | | Shine |
| Coat | Coat | Oil | Saliva | Acid | Adhesion | Shine | Retention |
| E | B | 1 | 1.06 | 1.06 | 1 | 64.2 | Top Coat |
| | | | | | | | Retained |
| E | C | 1 | 1 | 1 | 1 | 56 | Top Coat |
| | | | | | | | Removed |

Those skilled in the art will recognize or be able to ascertain using no more than routine experimentation, many equivalents to the specific embodiments of the invention described herein. Such equivalents are intended to be encompassed by the following claims.

1. A two-step reactive lip system comprising:
a base coat composition that comprises a silicone oil and a dispersion of copolymer particles surface-stabilized with one or more stabilizers in a non-aqueous medium containing at least one hydrocarbon-based oil, the copolymer consisting of a C₁-C₄ alkyl (meth)acrylate and maleic anhydride; and
a top coat composition that comprises at least one viscous silicone and a silicone amine, wherein the viscous silicone has a viscosity of at least about 100,000 centistoke when measured at a concentration by weight of 100% and using a rotational viscometer at 25° C.

2. The two-step reactive lip system according to claim 1, wherein the copolymer particles surface-stabilized with one or more stabilizers comprise:
between about 10% and about 30% by weight of the one or more stabilizers,
between about 50% and about 89.99% by weight of the C₁-C₄ alkyl (meth)acrylate, and
between about 0.01% and about 25% of the maleic anhydride.

3. The two-step reactive lip system according to claim 1, wherein the copolymer particles are present in an amount of between about 20% and about 60% by weight in the dispersion.

4. The two-step reactive lip system according to claim 1, wherein the copolymer comprises between 80% and 99.99% by weight of the C₁-C₄ alkyl (meth)acrylate and between 0.01% and 20% by weight of the maleic anhydride.

5. The two-step reactive lip system according to claim 1, wherein the hydrocarbon oil contains between 10 and 18 carbons.

6. The two-step reactive lip system according to claim 1, wherein the hydrocarbon oil is isodecane, isododecane, isohexadecane, n-dodecane, or n-tetradecane.

7. The two-step reactive lip system according to claim 1, wherein the dispersion is present in the base composition in an amount of between about 5% and about 75% by weight.

8. The two-step reactive lip system according to claim 1, wherein the viscous silicone is a dimethiconol.

9. The two-step reactive lip system according to claim 1, wherein the viscous silicone is present in a total amount of between about 0.1% and about 50% by weight in the top coat composition.

10. The two-step reactive lip system according to claim 1, wherein at least one pigment is present in a total amount of between 0.5% and 40% by weight in the base coat composition.

11. The two-step reactive lip system according to claim 1, wherein the silicone amine is present in a total amount of between 0.5% and 40% by weight in the top coat composition.

12. The two-step reactive lip system according to claim 1, wherein the one or more stabilizers comprises a (C₃-C₁₂)cycloalkyl (meth)acrylate polymer, the (C₃-C₁₂)cycloalkyl (meth)acrylate polymer being a (C₃-C₁₂)cycloalkyl (meth)acrylate homopolymer or a statistical copolymer of (C₃-C₁₂)cycloalkyl (meth)acrylate and of C₁-C₄ alkyl (meth)acrylate present in a (C₃-C₁₂)cycloalkyl (meth)acrylate/C₁-C₄ alkyl (meth)acrylate weight ratio of greater than 4.

13. The two-step reactive lip system according to claim 1, wherein the one or more stabilizers is isobornyl acrylate.

14. A method for providing long wear in a lip cosmetic, comprising the steps of:
applying to lips a base coat composition that comprises a silicone oil and a dispersion of copolymer particles surface-stabilized with one or more stabilizers in a non-aqueous medium containing at least one hydrocarbon-based oil, the copolymer consisting of a C₁-C₄ alkyl (meth)acrylate and maleic anhydride; and
applying a top coat composition that comprises at least one viscous silicone and a silicone amine on top of the base coat composition, wherein the viscous silicone has a viscosity of at least about 100,000 centistoke when measured at a concentration by weight of 100% and using a rotational viscometer at 25° C.

15. The method according to claim 14, wherein the copolymer particles surface-stabilized with one or more stabilizers comprise:
between about 10% and about 30% by weight of the one or more stabilizers,
between about 50% and about 89.99% by weight of the C₁-C₄ alkyl (meth)acrylate, and
between about 0.01% and about 25% by weight of the maleic anhydride.

16. The method according to claim 14, wherein the maleic anhydride is present in a total amount of between about 0.02% and about 8.4% by weight of the base coat composition.

17. The method according to claim 14, wherein the hydrocarbon oil contains between 10 and 18 carbons.

18. The method according to claim 14, wherein the dispersion is present in the base composition in an amount of between about 5% and about 75% by weight.

19. The method according to claim 14, wherein the viscous silicone is present in a total amount of between about 0.1% and about 50% by weight in the top coat composition.

20. The method according to claim 14, wherein the base coat composition further comprises at least one pigment.

21. The two-step reactive lip system according to claim 1, wherein the base coat composition, the top coat composition, or both further comprise a styrenic block copolymer.

22. The two-step reactive lip system according to claim 1, wherein the base coat composition, the top coat composition, or both further comprise a colorant.

23. The two-step reactive lip system according to claim 1, wherein the non-aqueous medium containing at least one hydrocarbon-based oil comprises at least two hydrocarbon-based oils.

24. The two-step reactive lip system according to claim 24, wherein the top coat composition further comprise a least one hydrocarbon-based oil.

25. (canceled)
